(19) United States
(12) Patent Application Publication
(10) Pub. No.: US 2024/0167984 A1
(43) Pub. Date: May 23, 2024

(54) METHODS FOR DETECTING AND DETERMINING PROTEIN STRUCTURES AND STABILITY IN FLUIDS, INCLUDING BIOLOGICAL FLUIDS

(71) Applicant: Regeneron Pharmaceuticals, Inc., Tarrytown, NY (US)

(72) Inventors: Michael Rosconi, New City, NY (US); Nina Liu, Chappaqua, NY (US); Erica Pyles, New City, NY (US)

(21) Appl. No.: 18/389,370

(22) Filed: Nov. 14, 2023

Related U.S. Application Data
(60) Provisional application No. 63/426,554, filed on Nov. 18, 2022.

(51) Int. Cl. G01N 30/00; G01N 33/58; G01N 33/68

(52) U.S. Cl. CPC G01N 30/0005; G01N 33/582; G01N 33/6854; G01N 2030/003

(57) ABSTRACT

The present inventions provide methods for detecting and determining protein structures and stability, including heteromeric protein complexes, in biological fluids, such as serum and other bodily fluids. Systems for performing the methods also are provided.

This application claims priority to U.S. Ser. No. 63/426,554, filed Nov. 18, 2022, which is hereby incorporated by reference.

### FIELD OF THE INVENTIONS

The present inventions provide methods for detecting and determining protein structures and stability, including heteromeric protein complex types in various fluids, including biological fluids such as serum and other bodily fluids. Systems for performing the methods also are provided.

### BACKGROUND OF THE INVENTIONS

Monoclonal antibody therapy is increasingly being used world-wide to treat medical disorders. When two types of non-competing monoclonal antibodies are present, both antibodies can bind to an antigenic entity, such as an infectious agent or fragment thereof, at different epitopes. FIG. 1 schematically depicts on the left side a ligand binding to monoclonal antibody 1 (mAb1), and on the right side a ligand binding with mAb1 and another antibody, mAb2. If mAb2 is in a significant molar excess compared to mAb1, small heteromeric complex types predominate. Where mAb1 and mAb2 approach molar equivalence, larger heteromeric complex types. In the body, such complexes can adversely impact safety and/or efficacy.

Asymmetric flow field-flow fractionation (A4F, also referred to as AF4) is a powerful tool for separating large molecules and large macromolecular complexes. See WO 2020/047067. Size exclusion chromatography (SEC) also is a powerful tool for separating large molecules and large macromolecular complexes.

A4F and SEC, however, must be paired with a detector, such as ultraviolet light (UV) and Multi-Angle Light Scattering (MALS), which is also known as Multi-Angle Laser Light Scattering (MALLS). These detectors, however, are not capable of detecting specific heteromeric protein complex types in biological fluids, such as serum, due to interference from other proteinaceous components that are typically present in these fluids.

Accordingly, there are needs to detect and determine heteromeric protein complex formation and types in various biological fluids. These needs were unmet until the present inventions.

### SUMMARY OF THE INVENTIONS

The inventions provide methods for characterizing one or more complex types comprising at least one protein of interest using asymmetric flow field-flow fractionation (A4F), wherein the method comprises the steps of: (A) fractionating a first aliquot comprising complexes in a fractionation buffer using A4F, and determining at least one of molar mass, stoichiometry, and size distribution of the complexes in the first aliquot using Multi-Angle Light Scattering (MALS); and (B) fractionating a second aliquot comprising the complexes bound with a fluorescence labeled detection reagent in a biological fluid using A4F, and detecting the protein of interest in the complexes using a fluorescence detector, wherein (A) and (B) can be performed consecutively in any order or simultaneously, and (C) comparing the elution time profiles from (A) and (B).

The protein of interest can be an Fc-containing protein, such as a monoclonal antibody or Fc-fusion protein. The first aliquot can be in a fractionation buffer and second aliquot can be in a biological fluid. The protein complex types can be monoclonal antibody (mAb)—ligand complexes. The second aliquot can comprise protein complexes in serum or various other biological fluids. The fluorescence labeled detection reagent can be a fluorescence labeled antibody or Fc-fusion protein, a fluorescence labeled anti-Fc Fab or a fluorescence labeled anti-antibody Fab, for example. The methods can determine stoichiometry of complex types in serum or various other biological fluids, masses of complex types in serum or various other biological fluids, and size distribution of complex types in serum or various other biological fluids. The second aliquot can be diluted in a fractionation buffer.

The inventions also provide methods for evaluation stability of a protein in a biological fluid using asymmetric flow field-flow fractionation (A4F), wherein the method comprises the steps of: (A) fractionating a first aliquot comprising the protein in a fractionation buffer using A4F, and determining size distribution of the protein in the first aliquot using Multi-Angle Light Scattering (MALS); (B) fractionating a second aliquot comprising the protein bound with a fluorescence labeled detection reagent in a biological fluid using A4F, and detecting the protein using a fluorescence detector, wherein (A) and (B) can be performed consecutively in any order or simultaneously. The fluorescence labeled detection reagent can be a fluorescence labeled protein, a fluorescence labeled anti-Fc Fab or a fluorescence labeled anti-protein Fab, for example.

The formation of high molecular weight species of the protein can indicate instability. The protein can be subjected to one or more freeze-thaw cycles prior to conducting step (A). The protein can be stored prior to conducting step (A), such as about 19 to 25° C. and/or at least about 1 to 60 days. The second aliquot can be diluted in a fractionation buffer.

The inventions also provide methods for characterizing one or more complex types comprising at least one protein of interest using fractionation, wherein the method comprises the steps of: (A) fractionating a first aliquot comprising the complexes in a fractionation buffer using asymmetric flow field-flow fractionation (A4F) or size exclusion chromatography (SEC) and determining at least one of molar mass, stoichiometry, and size distribution of the complexes in the first aliquot using Multi-Angle Light Scattering (MALS); (B) fractionating a second aliquot comprising the complexes bound with a fluorescence labeled detection reagent in a biological fluid using A4F or SEC, and detecting the protein of interest in the complexes using a fluorescence detector, wherein (A) and (B) can be performed consecutively in any order or simultaneously, and (C) comparing the elution time profiles from (A) and (B). Steps (A) and (B) can use A4F, or steps (A) and (B) can use SEC. The fluorescence labeled detection reagent can be a fluorescence labeled antibody, a fluorescence labeled anti-Fc Fab or a fluorescence labeled anti-antibody Fab, for example.

The stability of the complex types can be determined, such as where the formation of high molecular weight species of the protein indicates instability.

The second aliquot can be diluted in a fractionation buffer. The protein of interest can be a monoclonal antibody. The protein of interest can be an Fc-fusion protein, such as a receptor Fc-fusion.

The inventions also provide systems for performing any of the methods disclosed herein.

### BRIEF DESCRIPTION OF THE FIGURES

FIG. 1 schematically depicts on the left side a ligand binding to monoclonal antibody 1 (mAb1), and on the right side a ligand binding with mAb1 and another antibody, mAb2.

FIG. 2 schematically depicts immune detection reagents Fir-mAb2, Flr-anti-hFc Fab, and Fir-anti-mAb2 Fab. “Fir” indicates a fluorescent label. The schematic U-like structures indicate how Fir detection works.

FIG. 3 is a graph that depicts data from serum using a (i) UV detection and (ii) detection using an Fir-mAb2.

FIG. 4 is a graph that depicts data from 1*DPBS using mAb2:ligand and Fir-mAb2:ligand, and both achieve canonical 1:1 and 1:2 complex types with the ligand.

FIG. 5 is a graph that depicts data from 1*DPBS using mAb2:ligand and Fir-mAb2:ligand. The data show that both form large heterogeneous complex types with ligand and mAb1.

FIG. 6 schematically shows how (i) mAb2 and ligand is tested with A4F-MALS and (ii) Fir-mAb2 and ligand is tested with A4F-Flr. Each are tested in DPBS, ligand depleted human serum plus ligand, and native human serum, which has ligand already present.

FIG. 7 is a graph that depicts data showing that mAb2 and ligand form the same type of complex in DPBS buffer, ligand depleted serum and normal serum at 1.2 μM:0.4 μM in terms of mAb2:ligand.

FIG. 8 is a graph that depicts data showing that mAb2 and ligand form the same type of complex in DPBS buffer, ligand depleted serum and normal serum at 0.4 μM:0.4 μM in terms of mAb2:ligand

FIG. 9 is a graph that depicts data showing that mAb2 and ligand form the same type of complex in DPBS buffer, ligand depleted serum and normal serum at 0.2 μM:0.4 μM in terms of mAb2:ligand.

FIG. 10 is a graph that depicts data showing that mAb2, mAb1 and ligand form the same type of complex in DPBS buffer, ligand depleted serum and normal serum at 2 μM:0.4 μM:0.4 μM in terms of mAb2:mAb1:ligand.

FIG. 11 is a graph that depicts data showing that mAb2, mAb1 and ligand form the same type of complex in DPBS buffer, ligand depleted serum and normal serum at 0.2 μM:0.2 μM:0.4 μM in terms of mAb2:mAb1:ligand.

FIG. 12A is a chart that shows the theoretical molecular weights of mAb:anti-hFc Fab complex types. FIG. 12B contains graphs: (i) the top graph shows the peaks of anti-human Fc Fab and co-elution of mAb2 and mAb1 and (ii) the bottom graph shows anti-human Fc Fab and co-elution of 1:2 ratio complex types of mAb2:anti-human Fc Fab and mAb1:anti-human Fc Fab.

FIGS. 13A-13D collectively show that anti-hFc Fab binds to free monoclonal antibodies and monoclonal antibody ligand complex types. There are depicted analyses with mAb2:ligand (FIG. 13A), mAb2:mAb1:ligand (FIG. 13B), anti-hFc Fab:mAb2:ligand (FIG. 13C), and anti-hFc Fab:mAb2:mAb1:ligand (FIG. 13D).

FIG. 14 depicts formation of antibody-ligand complex types with different detection reagents in PBS, or mouse serum pre-treated with mAb2/mAb1.

FIG. 15 depicts use of the reagents of FIG. 14 to form complexes. The graphs show that at 0.9 μM:0.3 μM mAb2:ligand ratio, similar types of complexes are observed with either Flr-mAb2 or Flr-anti-hFc Fab as detection reagent in DPBS buffer, and with Flr-anti-hFc Fab as detection reagent in ex vivo mouse serum.

FIG. 16 depicts use of the reagents of FIG. 14 to form complexes. The graphs show that at 0.3 μM:0.3 μM mAb2:ligand ratio, similar types of complexes are observed with either Flr-mAb2 or Flr-anti-hFc Fab as detection reagent in DPBS buffer, and with Flr-anti-hFc Fab as detection reagent in ex vivo mouse serum.

FIG. 17 depicts use of the reagents of FIG. 14 to form complexes. The graphs show that at 0.15 μM:0.3 μM mAb2:ligand ratio, similar types of complexes are observed with Flr-mAb2 as detection reagent in DPBS buffer, and with Flr-anti-hFc Fab as detection reagent in DPBS buffer or ex vivo mouse serum.

FIG. 18 depicts use of the reagents of FIG. 14 to form complexes. The graph shows that at 1.5 μM:0.3 μM: 0.3 μM mAb2:mAb1:ligand ratio, similar types of complexes are observed with Flr-mAb2 as detection reagent in DPBS buffer, and with Flr-anti-hFc Fab as detection reagent in DPBS buffer or ex vivo mouse serum.

FIG. 19 depicts use of the reagents of FIG. 14 to form complexes. The graphs show that at 0.15 μM:0.15 μM:0.3 μM mAb2:mAb1:ligand ratio, similar types of complexes are observed with Flr-mAb2 as detection reagent in DPBS buffer, and with Flr-anti-hFc Fab as detection reagent in DPBS buffer or ex vivo mouse serum.

FIG. 20A is a chart that shows the theoretical molecular weights of mAb:anti-drug Fab complexes. Anti-drug Fab is a specific anti-mAb2 monoclonal IgG1 antibody. FIG. 20B is a graph based on A4F-MALS data that shows that anti-drug Fab can bind with free mAb2.

FIG. 21 is a graph based on A4F-MALS data that shows that anti-drug Fab can bind with mAb2 in mAb2:ligand complex.

FIG. 22 is a graph based on A4F-MALS data with two plots that shows that anti-drug Fab can bind with mAb2:mAb1:ligand complex types. Plot 1 uses 0.15 μM:0.15 μM:0.3 μM at ratios of mAb2:mAb1:ligand. Plot 2 uses 0.15 μM:0.15 μM:0.3 μM:0.45 μM at ratios of mAb2:mAb1:ligand:anti-drug Fab.

FIG. 23 depicts a schematic of the approach for formation of antibody-ligand complexes with anti-drug Fab in DPBS, and Flr-anti-drug Fab in mouse serum, ligand-depleted human serum formation and human serum.

FIG. 24 is a graph based on fluorescence data that shows that Flr-anti-drug Fab exhibits non-specific binding with human serum components.

FIG. 25 is a graph based on fluorescence data that shows that anti-drug Fab non-specific binding peak elutes earlier than the higher order complex types, and therefore does not affect complex detection in serum.

FIG. 26 is a graph that shows that mAb2 with ligand and Flr-anti-drug Fab at 2:1:1 anti-drug Fab:mAb2:ligand forms the same type of complex in DPBS buffer, mouse serum, ligand-depleted human serum and human serum. Ligand was present at 0.4 μM.

FIG. 27 is a graph that shows that mAb2 with ligand and Flr-anti-drug Fab at 1:0.5:0.5:1 anti-drug Fab:mAb2:mAb1:ligand forms the same type of complex in DPBS buffer, mouse serum, ligand-depleted human serum and human serum. Ligand was present at 0.4 μM.

FIG. 28 is a graph that shows that mAb2 with ligand and Flr-anti-drug Fab at 10:5:1:1 anti-drug Fab:mAb2:mAb1:ligand forms the same type of complex in DPBS buffer, mouse serum, ligand-depleted human serum and human serum. Ligand was present at 0.4 μM.

FIG. 29 is a graph based on data with two plots that show that Flr-anti-drug Fab can be used as a detection reagent to identify mAb2 containing higher order complex types in native human serum. Plot 1 used 1:0.5:0.5:1 Flr-anti-drug Fab:mAb2:mAb1:ligand. Plot 2 used 10:5:1:1 Flr-anti-drug Fab:mAb2:mAb1:ligand.

FIG. 30 depicts data from an A4F-MALS analysis that protein is stable in a storage buffer (10 mM sodium phosphate buffer and pH 6.0) following freeze/thaw (F/T), and more high molecular weight species were observed after stored at 25° C. for 5 days.

FIG. 31A is a graph depicting SEC-MALS that shows that an antiprotein monoclonal antibody can form canonical 1:1 and 1:2 complex types with the protein. FIG. 31B is a chart showing stoichiometry of complex types and theoretical molar mass.

FIGS. 32A and 32B are graphs showing that protein-antiprotein monoclonal antibody forms the same type of complex in PBS and mouse serum. FIG. 32A is a graph showing complex formation with 1 μM protein.

FIG. 32B is a graph showing complex formation with 0.08 μM protein. At both concentrations, protein is generally stable following freeze/thaw (F/T), more high molecular weight species were observed after stored at 25° C. for 5 days. The figures show that 0.08 μM protein (FIG. 32B) showed less high molecular weight species than 1 μM protein (FIG. 32A).

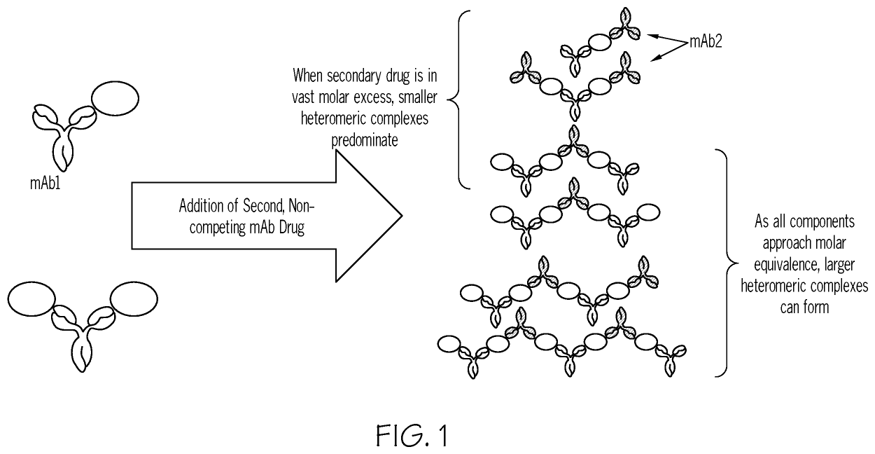

### DETAILED DESCRIPTION OF THE INVENTIONS

### Definitions

Unless defined otherwise, all technical and scientific terms used herein have the same meaning as commonly understood by one of ordinary skill in the art to which this invention belongs.

The term “about” in the context of numerical values and ranges refers to values or ranges that approximate or are close to the recited values or ranges such that the invention can perform as intended, such as having a desired rate, amount, density, degree, increase, decrease, percentage, value, purity, pH, concentration, presence of a form or variant, temperature or amount of time, as is apparent from the teachings contained herein. For example, “about” can signify values either above or below the stated value in a range of approx. +/−10% or more or less depending on the ability to perform. Thus, this term encompasses values beyond those simply resulting from systematic error.

“Biological fluid(s)” includes fluids obtained from the body, derived from fluids obtained from the body or contain biological materials, such as proteins. Examples include plasma, serum, cell media, aqueous humor, vitreous humor, interstitial fluid, lymph, synovial fluid, pleural fluid, pericardial fluid, peritoneal fluid and cerebrospinal fluid.

“Fluorescent labels” (“Flr”) provide a light signal that can be detected by a fluorescent detector, and can be bound to another molecule, such as an antibody, to form a fluorescence labeled detection reagent. Exemplary fluorescent labels are well-known in the art, including, but not limited to Discosoma coral (DsRed), green fluorescent protein (GFP), enhanced green fluorescent protein (eGFP), cyano fluorescent protein (CFP), enhanced cyano fluorescent protein (eCFP), yellow fluorescent protein (YFP), enhanced yellow fluorescent protein (eYFP) and far-red fluorescent protein (for example, mKate, mKate2, mPlum, m Raspberry or E2-crimson) as long as they do not interfere with complex formation. See, for example, U.S. Pat. Nos. 9,816,110. Alexa Fluor labels that are commercially-available and photostable also can be conjugated to antibodies. These labels are available with variety of absorption and emission capabilities.

A “fluorescence labeled detection reagent” comprises a fluorescent label (Fir) bound to an Fc-containing protein or a mini-trap protein. An example is an antibody bound to a fluorescent label, and generally referred to as an “Flr-labeled antibody” and other similar terminology.

A “Fractionation buffer” is a buffer a sample is placed and/or diluted in order to conduct fractionation using A4F or SEC. See WO 2020/047067. Buffers are readily attainable and commercially available. Proteins and protein complexes from any source, including biological sources, can be placed in a fractionation buffer.

“Antibodies” (also referred to as “immunoglobulins”) are examples of proteins having multiple polypeptide chains and extensive post-translational modifications. The canonical immunoglobulin protein (for example, IgG) comprises four polypeptide chains—two light chains and two heavy chains. Each light chain is linked to one heavy chain via a cysteine disulfide bond, and the two heavy chains are bound to each other via two cysteine disulfide bonds. Immunoglobulins produced in mammalian systems are also glycosylated at various residues (for example, at asparagine residues) with various polysaccharides, and can differ from species to species, which may affect antigenicity for therapeutic antibodies. Butler and Spearman, “The choice of mammalian cell host and possibilities for glycosylation engineering”, Curr. Opin. Biotech. 30:107-112 (2014).

Antibodies are often used as therapeutic biomolecules. An antibody includes immunoglobulin molecules comprised of four polypeptide chains, two heavy (H) chains and two light (L) chains inter-connected by disulfide bonds. Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDR1, LCDR2 and LCDR3. The term “high affinity” antibody refers to those antibodies having a binding affinity to their target of at least 10⁻⁹ M, at least 10⁻¹⁰ M; at least 10⁻¹¹ M; or at least 10⁻¹² M, as measured by surface plasmon resonance, for example, BIACORE™ or solution-affinity ELISA.

The phrase “bispecific antibody” includes an antibody capable of selectively binding two or more epitopes. Bispecific antibodies generally comprise two different heavy chains, with each heavy chain specifically binding a different epitope—either on two different molecules (for example, antigens) or on the same molecule (for example, on the same antigen). If a bispecific antibody is capable of selectively binding two different epitopes (a first epitope and a second epitope), the affinity of the first heavy chain for the first epitope will generally be at least one to two, three or four orders of magnitude lower than the affinity of the first heavy chain for the second epitope, and vice versa. The epitopes recognized by the bispecific antibody can be on the same or a different target (for example, on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions, and such sequences can be expressed in a cell that expresses an immunoglobulin light chain. A typical bispecific antibody has two heavy chains each having three heavy chain CDRs, followed by (N-terminal to C-terminal) a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding or one or both of the heavy chains to one or both epitopes.

The phrase “heavy chain,” or “immunoglobulin heavy chain” includes an immunoglobulin heavy chain constant region sequence from any organism, and unless otherwise specified includes a heavy chain variable domain. Heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a CH1 domain, a hinge, a CH2 domain, and a CH3 domain. A functional fragment of a heavy chain includes a fragment that is capable of specifically recognizing an antigen (for example, recognizing the antigen with a KD in the micromolar, nanomolar, or picomolar range), that is capable of expressing and secreting from a cell, and that comprises at least one CDR.

The phrase “light chain” includes an immunoglobulin light chain constant region sequence from any organism, and unless otherwise specified includes human kappa and lambda light chains. Light chain variable (VL) domains typically include three light chain CDRs and four framework (FR) regions, unless otherwise specified. Generally, a full-length light chain includes, from amino terminus to carboxyl terminus, a VL domain that includes FR1-CDR1-FR2-CDR2-FR3-CDR3-FR4, and a light chain constant domain. Light chains that can be used with these inventions include those, for example, that do not selectively bind either the first or second antigen selectively bound by the antigen-binding protein. Suitable light chains include those that can be identified by screening for the most commonly employed light chains in existing antibody libraries (wet libraries or in silico), where the light chains do not substantially interfere with the affinity and/or selectivity of the antigen-binding domains of the antigen-binding proteins. Suitable light chains include those that can bind one or both epitopes that are bound by the antigen-binding regions of the antigen-binding protein.

The phrase “variable domain” includes an amino acid sequence of an immunoglobulin light or heavy chain (modified as desired) that comprises the following amino acid regions, in sequence from N-terminal to C-terminal (unless otherwise indicated): FRI, CDRI, FR2, CDR2, FR3, CDR3, FR4. A “variable domain” includes an amino acid sequence capable of folding into a canonical domain (VH or VL) having a dual beta sheet structure wherein the beta sheets are connected by a disulfide bond between a residue of a first beta sheet and a second beta sheet.

The phrase “complementarity determining region” (“CDR”) includes an amino acid sequence encoded by a nucleic acid sequence of an organism's immunoglobulin genes that normally (that is, in a wild-type organism) appears between two framework regions in a variable region of a light or a heavy chain of an immunoglobulin molecule (for example, an antibody or a T cell receptor). A CDR can be encoded by, for example, a germline sequence or a rearranged or unrearranged sequence, and, for example, by a naive or a mature B cell or a T cell. In some circumstances (for example, for a CDR3), CDRs can be encoded by two or more sequences (for example, germline sequences) that are not contiguous (for example, in a nucleic acid sequence that has not been rearranged) but are contiguous in a B cell nucleic acid sequence, for example, as the result of splicing or connecting the sequences (for example, V-D-J recombination to form a heavy chain CDR3).

“Antibody derivatives and fragments” include, but are not limited to: antibody fragments (for example, ScFv-Fc, dAB-Fc, half antibodies, Fab), multispecifics (for example, IgG-ScFv, IgG-dab, ScFV-Fc-ScFV, tri-specific). “Fab” refers to an antibody fragment comprising an antigen binding region. An Fab typically will lack the Fc portion.

The phrase “Fc-containing protein” includes antibodies, bispecific antibodies, antibody derivatives containing an Fc, antibody fragments containing an Fc, Fc-fusion proteins, receptor Fc-fusion proteins (including trap proteins), immunoadhesins, and other binding proteins that comprise at least a functional portion of an immunoglobulin CH2 and CH3 region. A “functional portion” refers to a CH2 and CH3 region that can bind a Fc receptor (for example, an FcyR; or an FcRn, (neonatal Fc receptor), and/or that can participate in the activation of complement. If the CH2 and CH3 region contains deletions, substitutions, and/or insertions or other modifications that render it unable to bind any Fc receptor and also unable to activate complement, the CH2 and CH3 region is not functional. Fc-fusion proteins include, for example, Fc-fusion (N-terminal), Fc-fusion (C-terminal), mono-Fc-fusion and bispecific Fc-fusion proteins.

“Fc” stands for fragment crystallizable, and is often referred to as a fragment constant. Antibodies contain an Fc region that is made up of two identical protein sequences. IgG has heavy chains known as γ-chains. IgA has heavy chains known as α-chains, IgM has heavy chains known as μ-chains. IgD has heavy chains known as α-chains. IgE has heavy chains known as ε-chains. In nature, Fc regions are the same in all antibodies of a given class and subclass in the same species. Human IgGs have four subclasses and share about 95% homology amongst the subclasses. In each subclass, the Fc sequences are the same. For example, human IgG1 antibodies will have the same Fc sequences. Likewise, IgG2 antibodies will have the same Fc sequences; IgG3 antibodies will have the same Fc sequences; and IgG4 antibodies will have the same Fc sequences. Alterations in the Fc region create charge variation.

Fc-containing proteins, such as antibodies, can comprise modifications in immunoglobulin domains, including where the modifications affect one or more effector function of the binding protein (for example, modifications that affect FcyR binding, FcRn binding and thus half-life, and/or CDC activity). Such modifications include, but are not limited to, the following modifications and combinations thereof, with reference to EU numbering of an immunoglobulin constant region: 238, 239, 248, 249, 250, 252, 254, 255, 256, 258, 265, 267, 268, 269, 270, 272, 276, 278, 280, 283, 285, 286, 289, 290, 292, 293, 294, 295, 296, 297, 298, 301, 303, 305, 307, 308, 309, 311, 312, 315, 318, 320, 322, 324, 326, 327, 328, 329, 330, 331, 332, 333, 334, 335, 337, 338, 339, 340, 342, 344, 356, 358, 359, 360, 361, 362, 373, 375, 376, 378, 380, 382, 383, 384, 386, 388, 389, 398, 414, 416, 419, 428, 430, 433, 434, 435, 437, 438, and 439.

For example, and not by way of limitation, the binding protein is an Fc-containing protein (for example, an antibody) and exhibits enhanced serum half-life (as compared with the same Fc-containing protein without the recited modification(s)) and have a modification at position 250 (for example, E or Q); 250 and 428 (for example, L or F); 252 (for example, L/Y/F/W or T), 254 (for example, S or T), and 256 (for example, S/R/Q/E/D or T); or a modification at 428 and/or 433 (for example, L/R/SI/P/Q or K) and/or 434 (for example, H/F or Y); or a modification at 250 and/or 428; or a modification at 307 or 308 (for example, 308F, V308F), and 434. In another example, the modification can comprise a 428L (for example, M428L) and 434S (for example, N434S) modification; a 428L, 2591 (for example, V259I), and a 308F (for example, V308F) modification; a 433K (for example, H433K) and a 434 (for example, 434Y) modification; a 252, 254, and 256 (for example, 252Y, 254T, and 256E) modification; a 250Q and 428L modification (for example, T250Q and M428L); a 307 and/or 308 modification (for example, 308F or 308P).

“Fv” stands for fragment variable, and is primarily responsible for binding to epitopes.

All numerical limits and ranges set forth herein include all numbers or values thereabout or there between of the numbers of the range or limit. The ranges and limits described herein expressly denominate and set forth all integers, decimals and fractional values defined and encompassed by the range or limit. Thus, a recitation of ranges of values herein are merely intended to serve as a way of referring individually to each separate value falling within the range, unless otherwise indicated herein, and each separate value is incorporated into the specification as if it were individually recited herein.

### DESCRIPTION

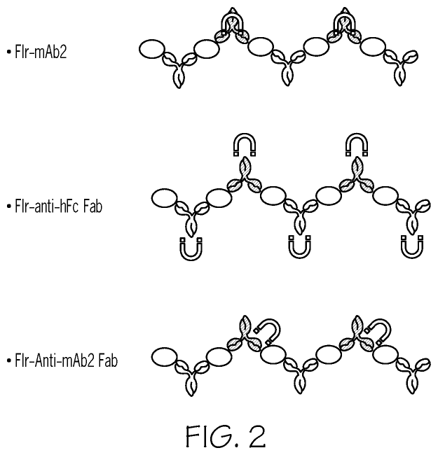

The present inventions combine A4F with a Fluorescent (Fir) detector to advantageously detect heteromeric protein complex types in biological fluids. Exemplary fluorescent detection reagents for evaluation immunological reactions are depicted in FIG. 2. In FIG. 2, the schematic U-like structures indicate how Fir detection works. The top representation depicts Fir-labeled shaded antibody (mAb2). The middle representation depicts that Fir-labeled anti-hFc Fab will bind to both the shaded and the not-shaded antibodies. The bottom representation depicts that Fir-labeled anti-mAb2 Fab will bind the shaded antibody.

For the experiments described herein, any fluorescent label with excitation and emission wavelength greater than 550 nm is preferred, such as Alexa Fluor™ 647.

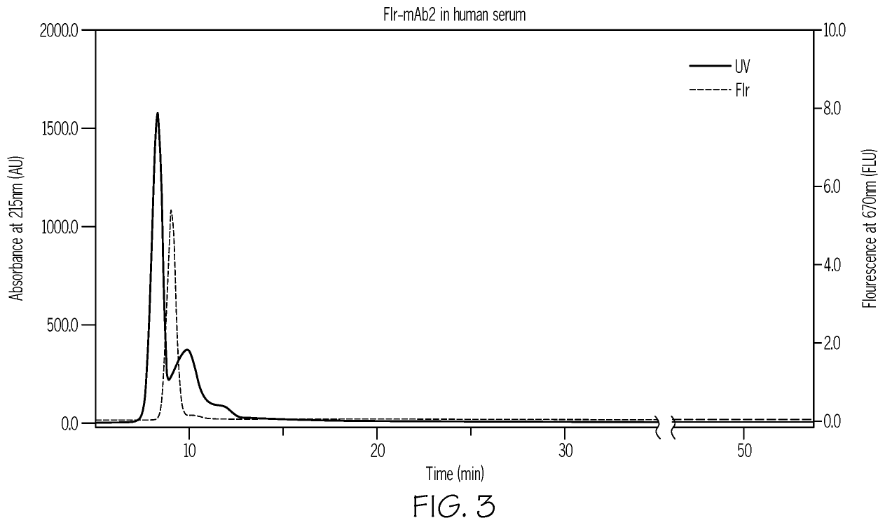

The superiority of Fir detection over UV detection is depicted in FIG. 3. Using a fluorescent labeled monoclonal antibody (Fir-mAb2), was able to detect an antibody of interest specifically, whereas a UV detector shows the total protein distribution and is not suitable to detect a specific protein in serum.

A4F combined with MALS (A4F-MALS) according to the inventions is a powerful methodology to detect and determine protein complex types in biological fluids. The invention can be used for stoichiometric analysis of ligand-mAb complexes in serum and protein stability in serum. An A4F fractionation buffer can be employed, such as DPBS, pH 7.4 buffer or 10 mM sodium phosphate, 500 mM sodium chloride, pH 7.0

Flr-labeled humanized monoclonal antibodies were first evaluated to determine if they behaved similarly to native humanized monoclonal antibodies. Here, the humanized monoclonal antibody was an anti-C5 antibody.

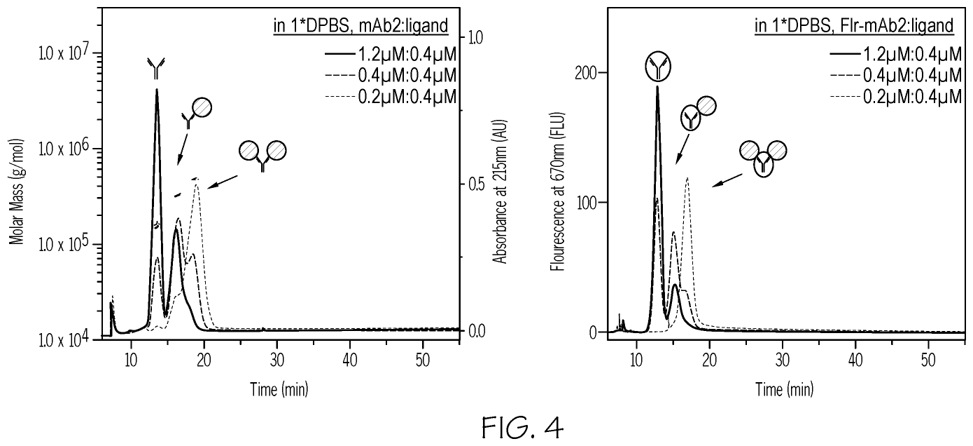

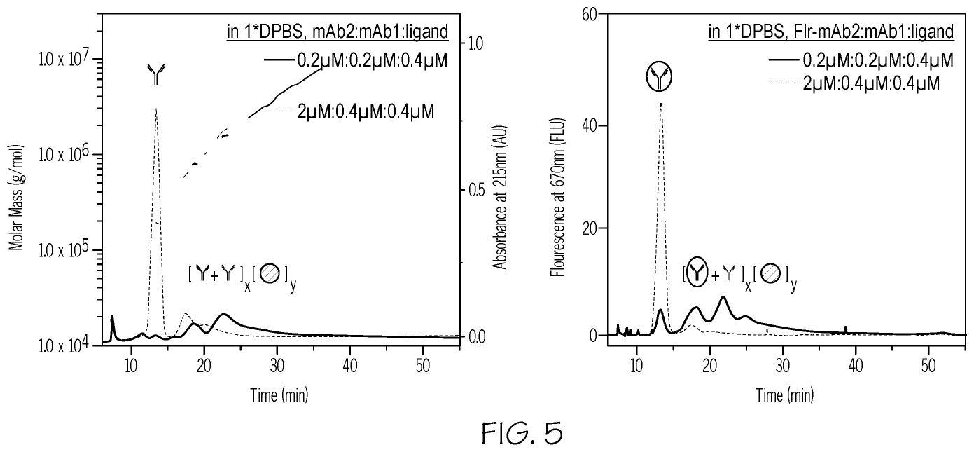

FIG. 4 depicts data from 1*DPBS (Dulbecco's phosphate buffered saline) using mAb2:ligand (Y-axis is molar mass) and Flr-mAb2:ligand (Y-axis is fluorescence). Both mAb2:ligand and Flr-mAb2:ligand at respective antibody to ligand concentration ratios 1.2 μM:0.4 μM, 0.4 μM:0.4 μM and 0.2 μM:0.4 μM achieve canonical 1:1 and 1:2 complex types with the ligand. Similarly, in FIG. 5, mAb2 and Flr-mAb2 behaved similarly with mAb1 and ligand in forming large, heterogeneous complex types. The tested concentration ratios of mAb2 and Flr-mAb2 to mAb1 and ligand were 0.2 μM:0.2 μM:0.4 μM and 2 μM:0.4 μM:0.4 μM. The data established that Flr-labeled monoclonal antibodies behave similarly to the native monoclonal antibodies upon which the Flr-labeled monoclonal antibodies are based. Accordingly, Flr-labeled monoclonal antibodies will be able to accurately detect and be useful to determine phenomena that occur in biological fluids.

The inventions are further described by the following Examples, which do not limit the inventions in any manner. The order of performance of the below examples can be altered or combined as determined by the person of skill in the art.

### Example 1—Analysis of Antibody Ligand Complex Types Using Flr-mAb2 in DPBS, Ligand-Depleted Serum Plus Ligand and Human Serum

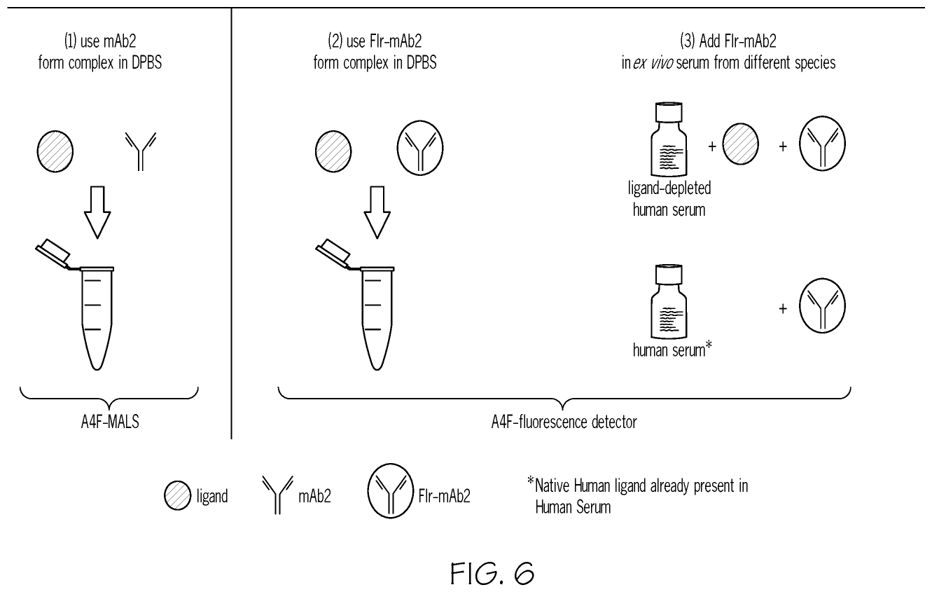

FIG. 6 schematically shows how (i) mAb2 and ligand is tested with A4F-MALS and (ii) Flr-mAb2 and ligand is tested with A4F and a fluorescence detector (A4F-Flr). Each are tested in DPBS, ligand depleted human serum plus ligand, and native human serum, which has ligand already present.

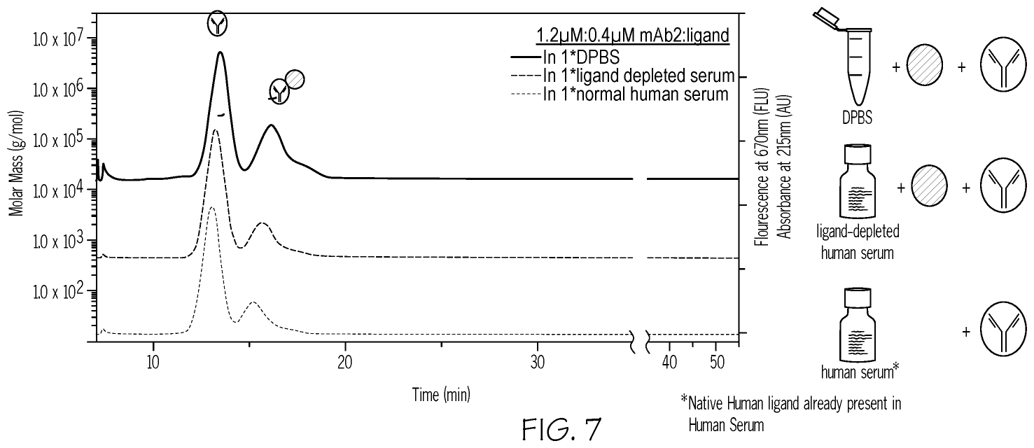

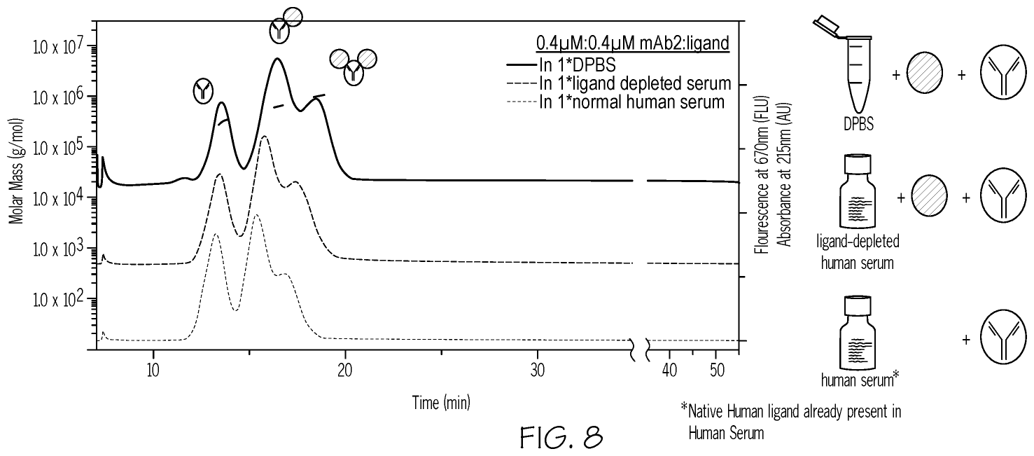

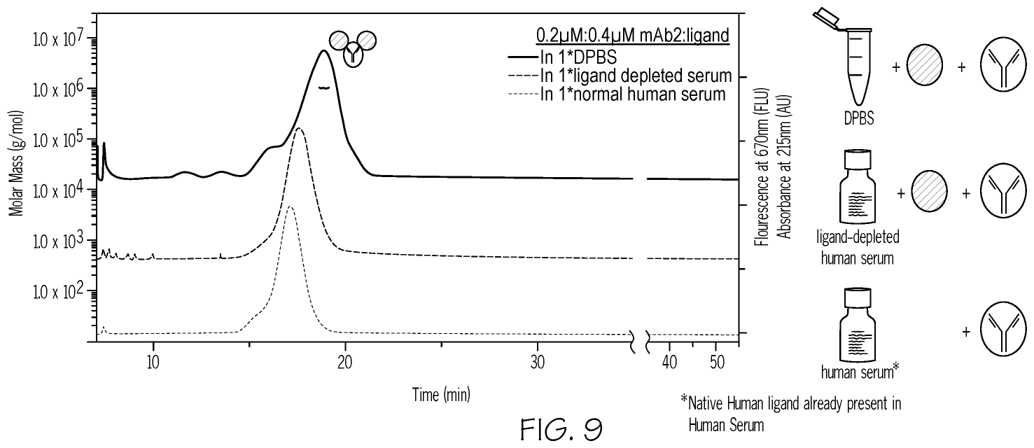

FIG. 7 is a graph that depicts data showing that mAb2 and ligand form the same type of complex in DPBS buffer, ligand depleted serum and normal serum at 1.2 μM:0.4 μM in terms of mAb2:ligand. Similarly, FIG. 8 is a graph that depicts data showing that mAb2 and ligand form the same type of complex in DPBS buffer, ligand depleted serum and normal serum at 0.4 μM:0.4 μM in terms of mAb2:ligand Again similar, FIG. 9 is a graph that depicts data showing that mAb2 and ligand form the same type of complex in DPBS buffer, ligand depleted serum and normal serum at 0.2 μM:0.4 μM in terms of mAb2:ligand. This data was obtained with A4F-MALS (top trace using DPBS) and A4F-Flr (bottom trace using serum).

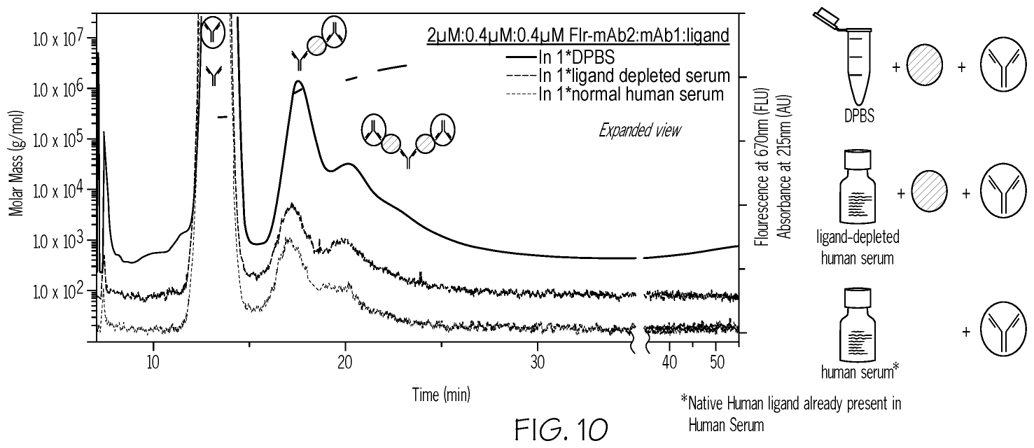

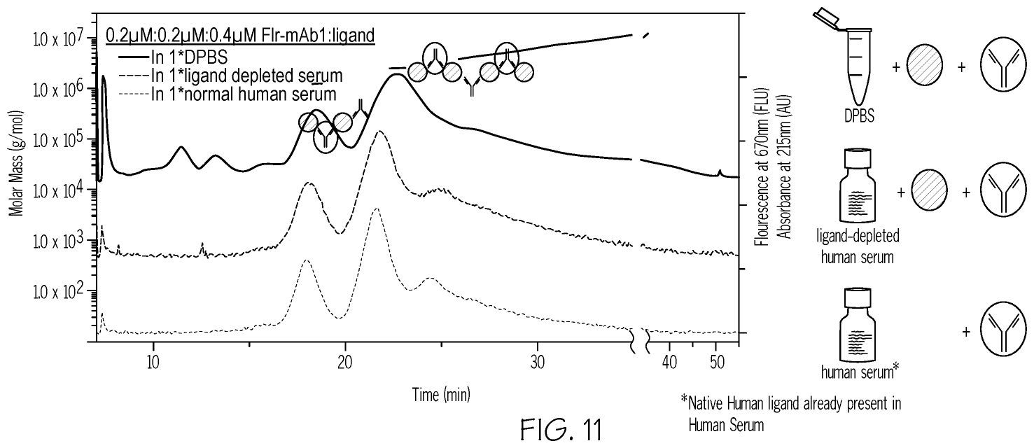

FIG. 10 is a graph that depicts data showing that mAb2, mAb1 and ligand form the same type of complex in DPBS buffer, ligand depleted serum and normal serum at 2 μM:0.4 μM:0.4 μM in terms of mAb2:mAb1:ligand. Similarly, FIG. 11 is a graph that depicts data showing that mAb2, mAb1 and ligand form the same type of complex in DPBS buffer, ligand depleted serum and normal serum at 0.2 μM:0.2 μM:0.4 μM in terms of mAb2:mAb1:ligand.

### Example 2—Analysis of Antibody Ligand Complex Types Using Fir Anti-Human Fc Fab in DPBS, Ligand-Depleted Serum Plus Ligand, and Human Serum

The immune detection reagents should not be competitive with ligand, and should not induce large heteromeric complex types, so that the Fir labeling cannot impact complex formation. A4F-MALS indicated that anti-human Fc Fab can bind to mAb2 (IgG4) and mAb1 (IgG4). With an excess amount of anti-human Fc Fab, each monoclonal antibody can bind up to a 2 molecules of anti-human Fc Fab.

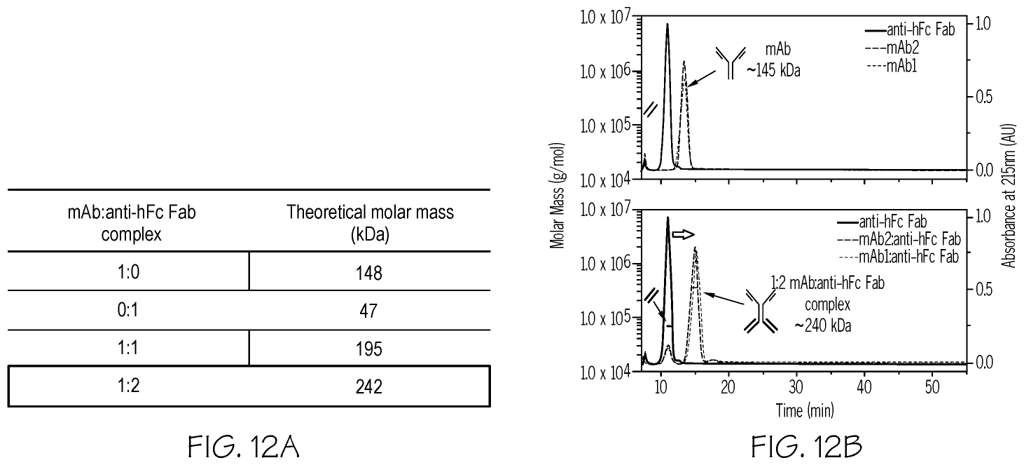

FIG. 12A is a chart that shows the theoretical molecular weights of mAb:anti-hFc Fab complex types. FIG. 12B contains two graphs: (i) the top graph shows the peaks of anti-human Fc Fab and co-elution of mAb2 and mAb1 and (ii) the bottom graph shows anti-human Fc Fab and co-elution of 1:2 ratio complex types of mAb2:anti-human Fc Fab and mAb1:anti-human Fc Fab.

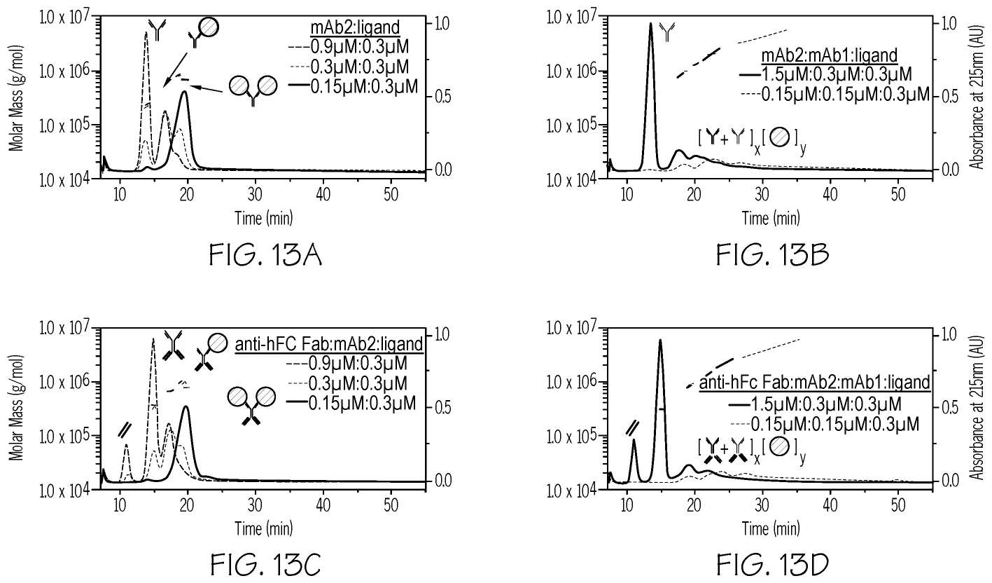

FIGS. 13A-D collectively show that anti-hFc Fab binds to free monoclonal antibodies and monoclonal antibody ligand complexes. There are depicted analyses with mAb2:ligand at ratios of 0.9 μM:0.3 μM, 0.3 μM:0.3 μM and 0.15 μM:0.3 μM (FIG. 13A), mAb2:mAb1:ligand at ratios of 1.5 μM:0.3 μM:0.3 μM and 0.15 μM:0.15 μM:0.3 μM (FIG. 13B), anti-hFc Fab binds to mAb2:mAb1:ligand at ratios of 0.9 μM:0.3 μM, 0.3 μM:0.3 μM and 0.15 μM:0.3 μM (FIG. 13C), and anti-hFc Fab binds to mAb2:mAb1:ligand at ratios of 1.5 μM:0.3 μM:0.3 μM and 0.15 μM:0.15 μM:0.3 μM (FIG. 13D).

These experiments served as a positive control. As shown, mAb2/ligand and mAb2/mAb1/ligand form the same type of complex without anti-hFc Fab (FIGS. 13A and 13C) and with anti-hFc Fab (FIGS. 13B and 13D). Therefore, this Fab can be used as detection reagent for mAb2.

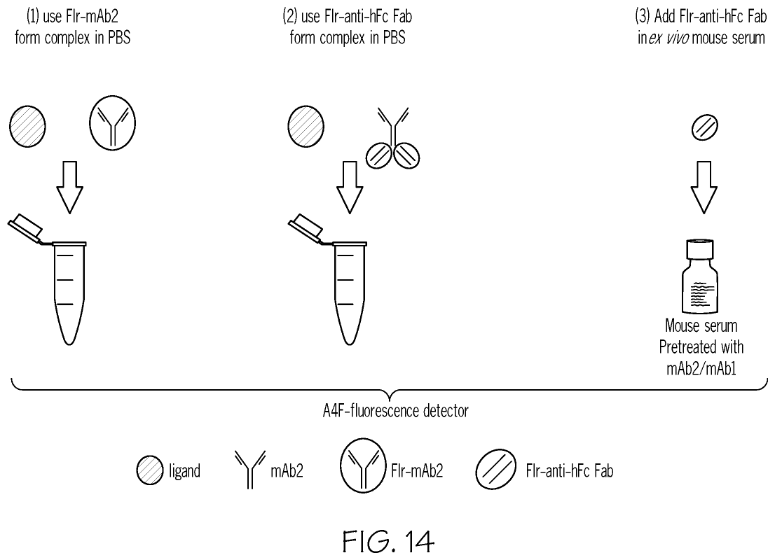

A summary of antibody-ligand complex formation with different detection reagents in PBS or ex vivo mouse serum pre-treated with mAb2/mAb1 is shown in FIG. 14. An A4F fluorescence detector is used to detect binding of ligand to mAb1 and mAb2 using Flr-mAb2 or Flr-anti-hFc Fab (which can bind to mAb1 and mAb2). Specific experiments are described below.

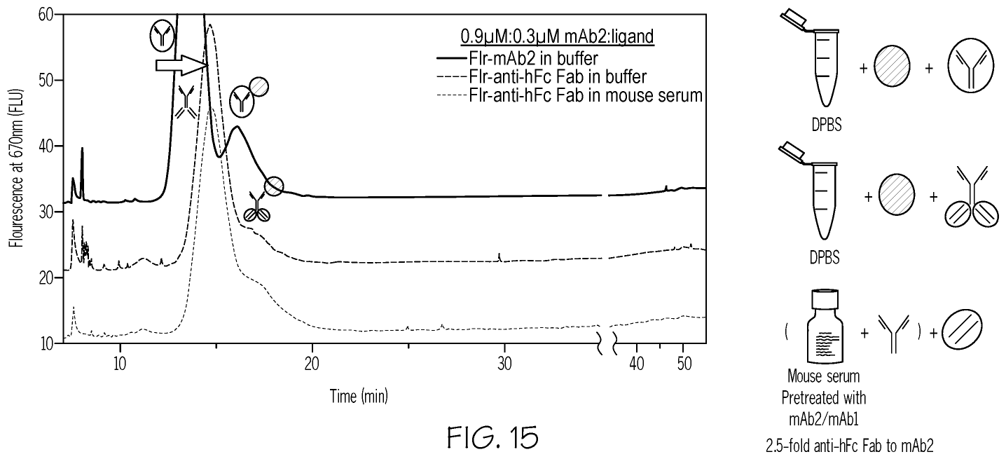

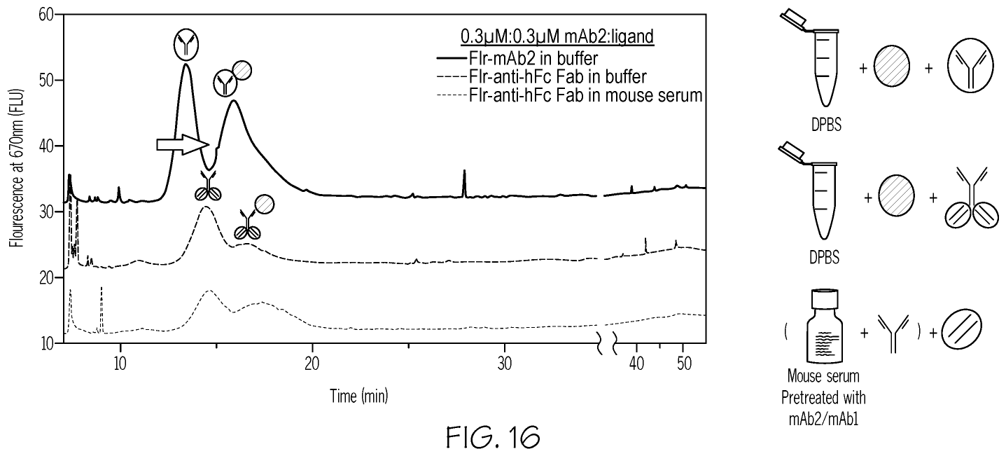

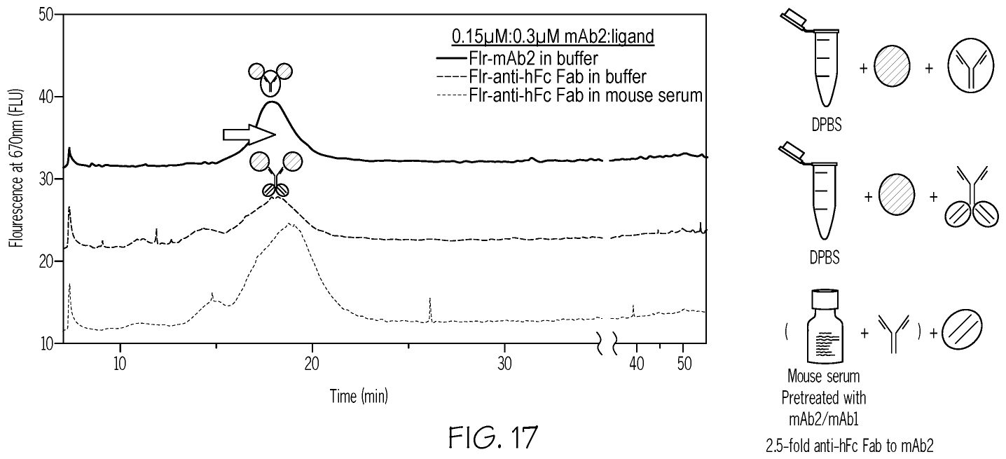

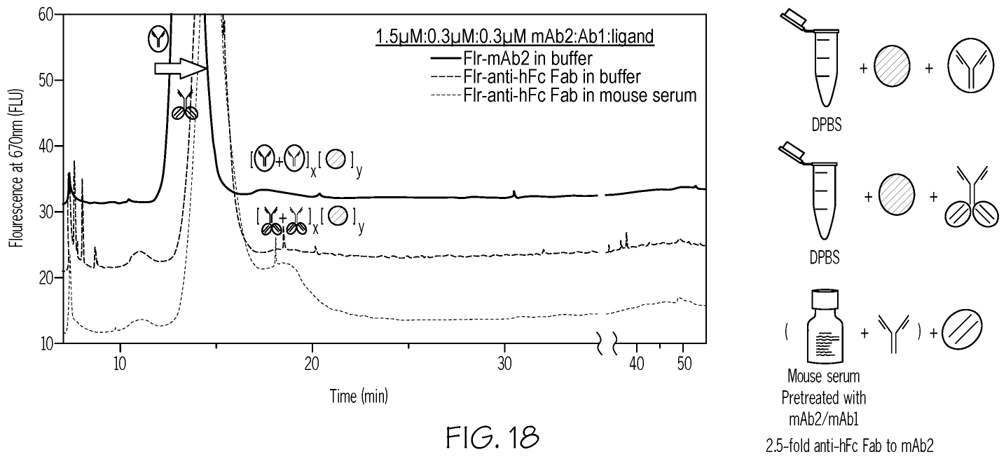

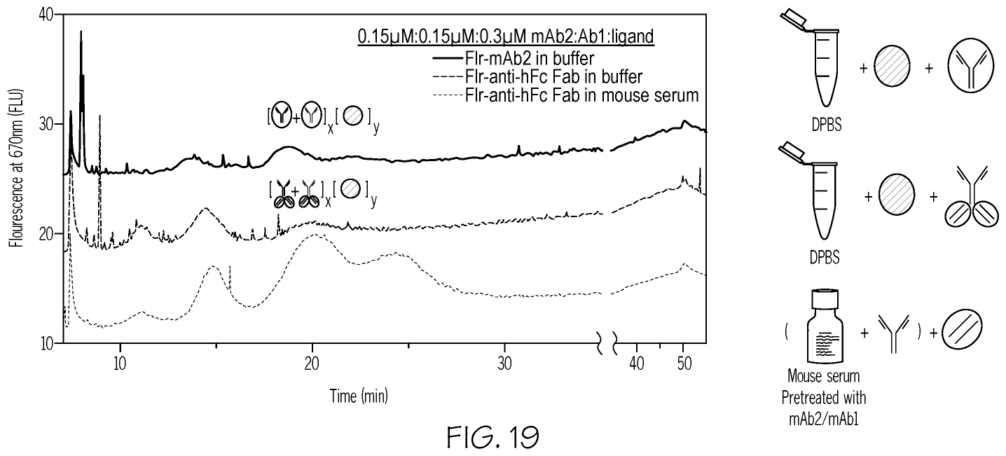

FIGS. 15, 16, 17, and 18 depict use of the reagents from FIG. 14, but in different molar ratios, in order to form complexes. The graphs contained in these figures show that mAb2 forms the same type of complex with ligand using Fir-mAb2 as detection reagent in DPBS buffer, and using Flr-anti-hFc Fab as detection reagent in Buffer or in ex vivo mouse serum. FIG. 15 used a concentration of 0.9 μM:0.3 μM mAb2:ligand. FIG. 16 used a concentration of 0.3 μM:0.3 μM mAb2:ligand. FIG. 17 used a concentration of 0.15 μM:0.3 μM mAb2:ligand. FIG. 18 used a concentration of 1.5 μM:0.3 μM mAb2:mAb1:ligand. FIG. 19 used a concentration of 0.15 μM:0.15 μM:0.3 μM mAb2:mAb1:ligand. In each, Flr-anti-hFc Fab was in a 2.5 fold molar excess to the concentration of mAb2.

The data show that complex formation was consistent between using Fir-mAb2 and using Flr-anti-hFc Fab at each concentration, and the A4F-Flr is capable of detecting stoichiometry of complex types in serum.

### Example 3—Analysis of Antibody Ligand Complex Types Using Flr-Anti-Drug Fab in DPBS, Ligand-Depleted Serum Plus Ligand, and Human Serum

Fir anti-hFc Fab can bind selectively to human IgG in mouse serum. However, human serum contains about 15 mg/mi (about 100 μM) of human IgG, and thus Fir anti-hFc Fab cannot bind selectively to the human antibody mAb2. Thus, the challenge is how to detect complexes comprising human mAb2 in human serum that contains human IgG. A different immune detection reagent had to be developed that was specific to mAb2, would not be competitive with the ligand, and would not induce large heteromeric complex formation.

The inventions therefore provide an anti-drug Fab that is a specific anti-mAb2 from a monoclonal mouse IgG1 antibody. In order to limit heteromeric complex formation with mAb2, the Fab portion of the anti-drug mAb was generated with a Ficin digestion followed by Protein G purification. Ficin is used to digest anti-mAb2 antibody so Fab and Fc will be separated. Protein G is used to enrich Fab from the digestion reaction.

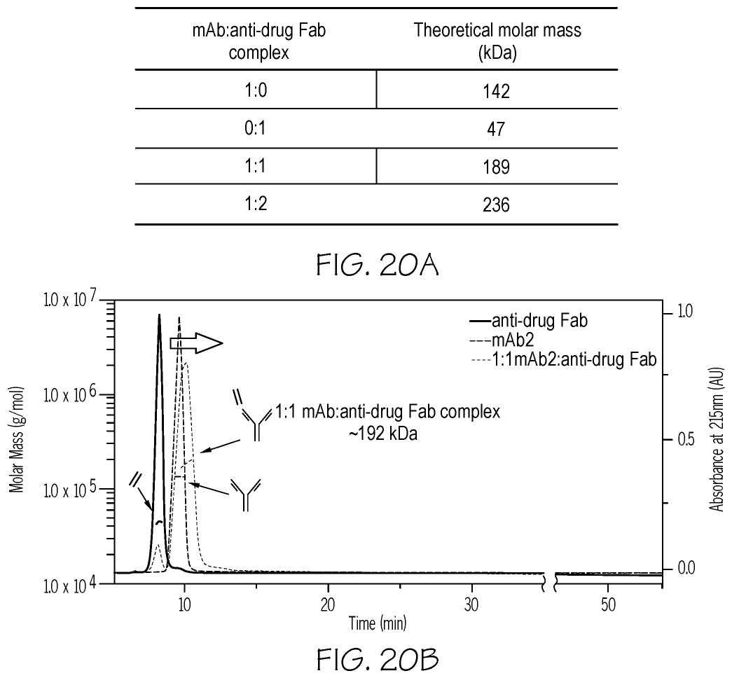

FIG. 20A is a chart that shows the theoretical molecular weights of mAb:anti-drug Fab complex types. FIG. 20B is a graph based on A4F-MALS data that shows that anti-drug Fab can bind with free mAb2, and the binding results in increases in the retention time and molar mass.

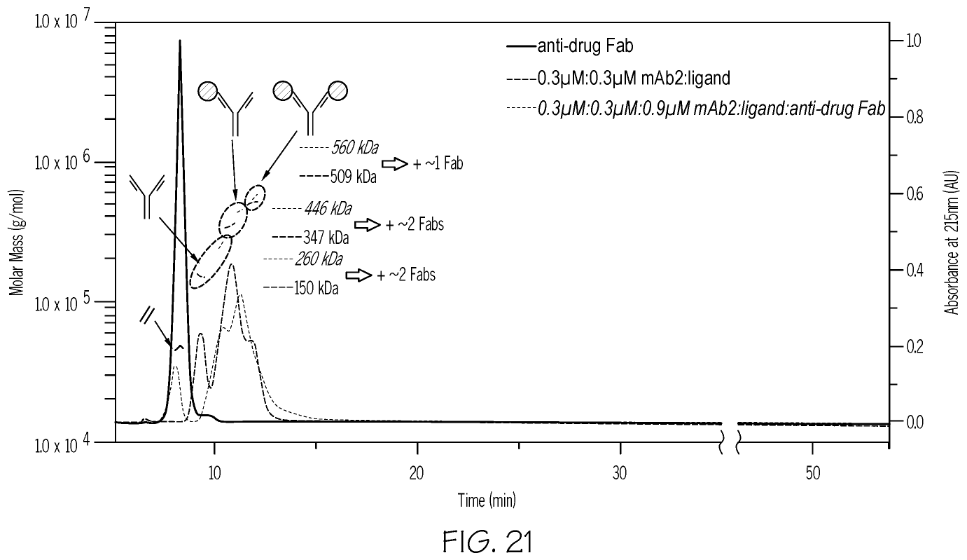

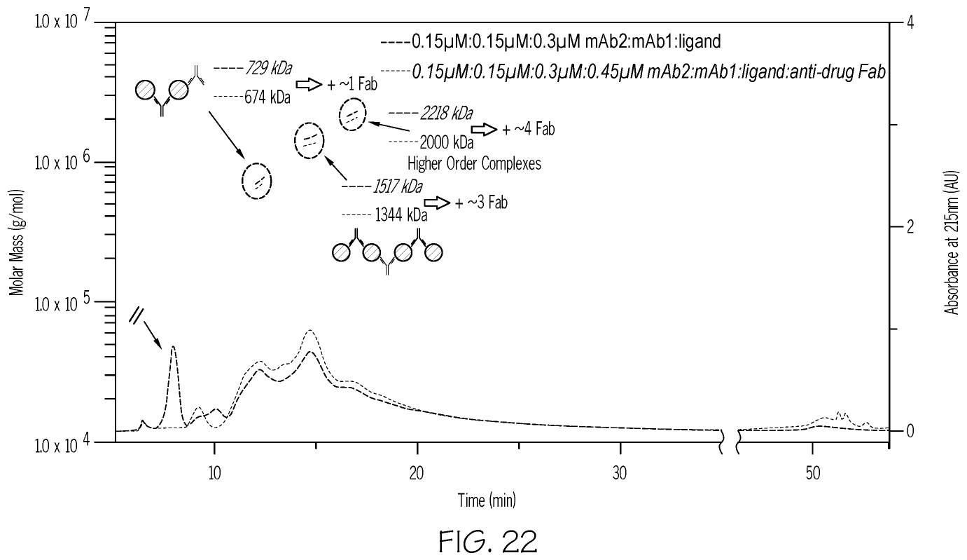

FIG. 21 is a graph based on A4F-MALS data that shows that anti-drug Fab can bind with mAb2 in mAb2:ligand complex, and the binding results in increases in the retention time and molar mass. FIG. 22 is a graph based on A4F-MALS data with two plots that shows that anti-drug Fab can bind with mAb2 in mAb2:mAb1:ligand complex types. Plot 1 uses 0.15 μM:0.15 μM:0.3 μM at ratios of mAb2:mAb1:ligand. Plot 2 uses 0.15 μM:0.15 μM:0.3 μM:0.45 μM at ratios of mAb2:mAb1:ligand:anti-drug Fab. Although higher order complex types show little to no elution shift, an increase in molar mass can be observed that is indicative of Fab binding.

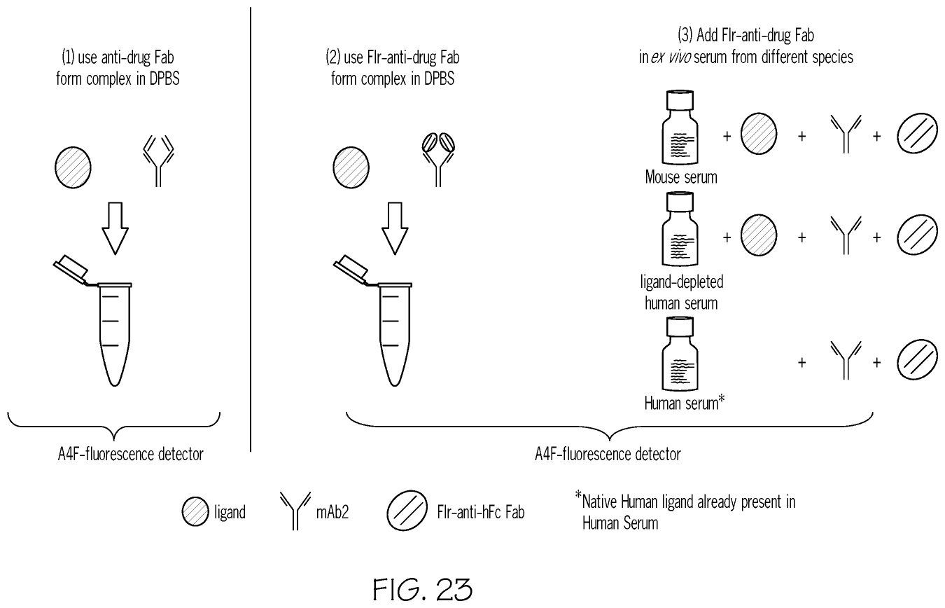

A schematic of the approach for formation of antibody-ligand complexes with anti-drug Fab in DPBS, and Flr-anti-drug Fab in mouse serum, ligand-depleted human serum and human serum is set forth in FIG. 23. The anti-drug Fab is analysed with A4F-MALS, and the Flr-anti-drug Fab was tested with A4F-Flr. Note that the native human ligand already is present in the human serum, designated by an asterisk (*).

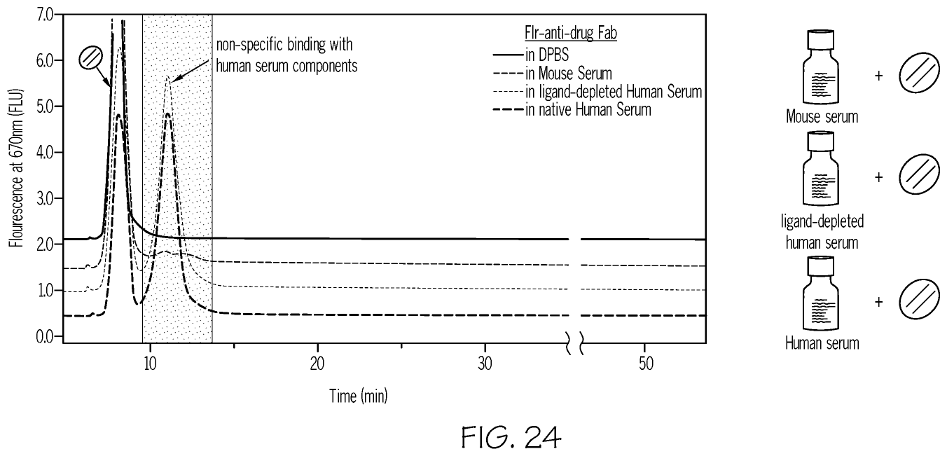

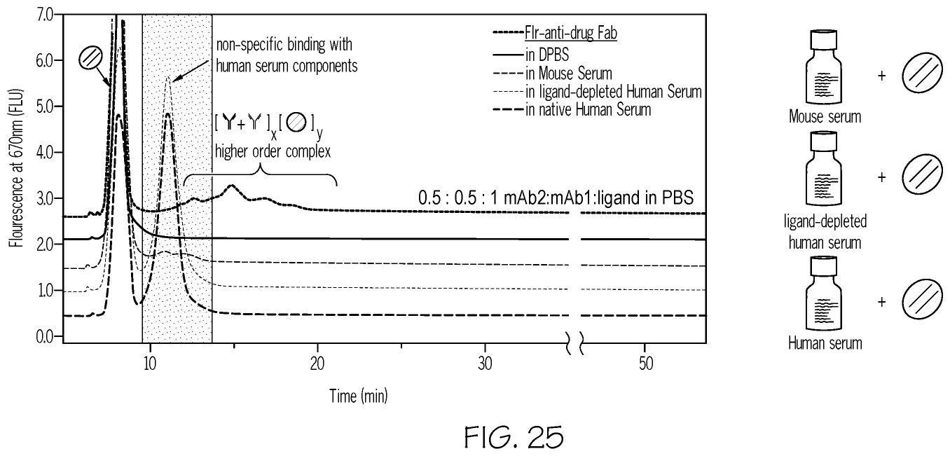

FIG. 24 shows that with fluorescence detection the Flr-anti-drug Fab exhibits non-specific binding with human serum components. However, as shown in FIG. 25, anti-drug Fab non-specific binding peak elutes earlier than the higher order complex types as detected with fluorescence, and therefore does not interfere complex detection in serum.

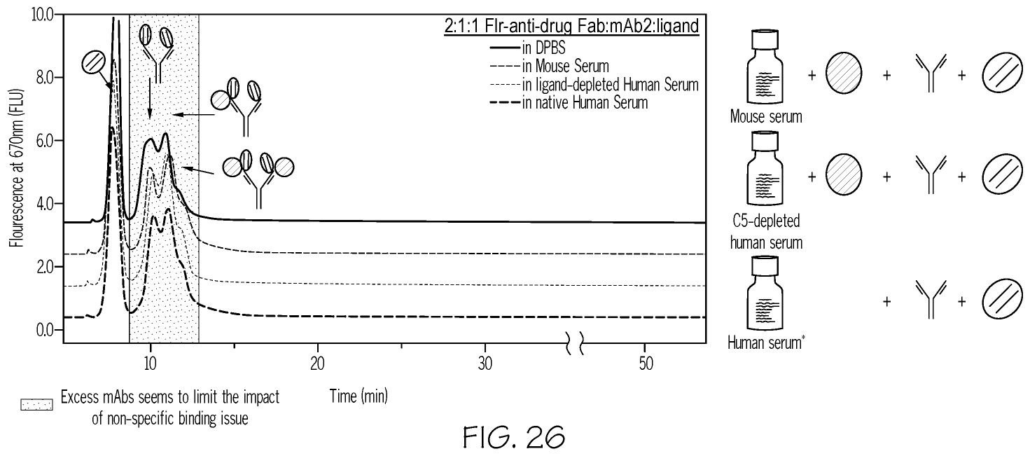

As depicted in FIG. 26, mAb2 with ligand and Flr-anti-drug Fab at a 2:1:1 ratio of Flr-anti-drug Fab:mAb2:ligand forms the same type of complexes in DPBS buffer, mouse serum, ligand-depleted human serum and human serum. Ligand was present at 0.4 μM. Excess mAb2 appears to limit the impact of non-specific binding.

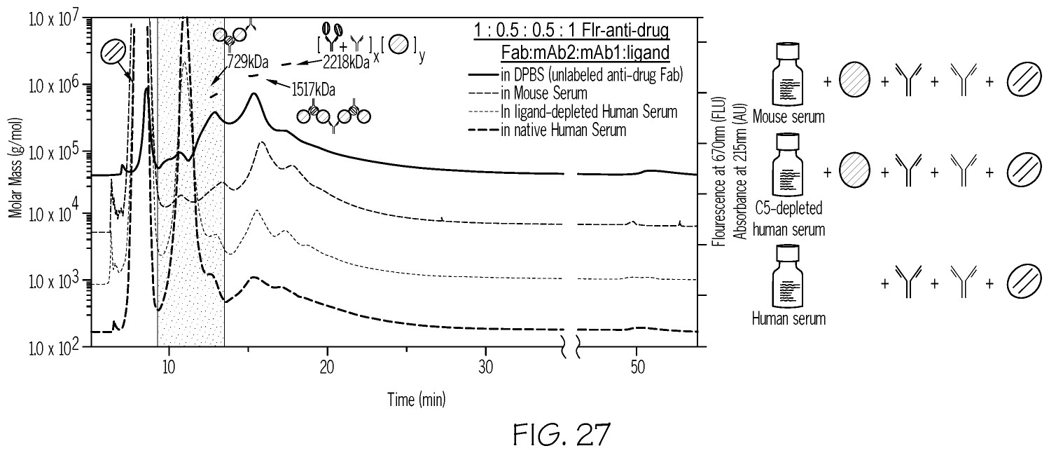

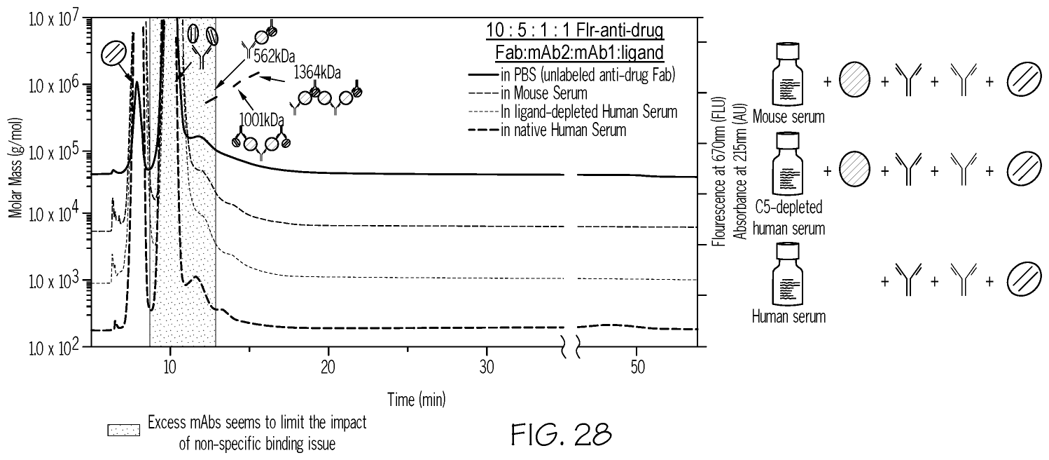

The types of observed complex types were consistent within each test ratio in DPBS buffer, mouse serum, ligand-depleted human serum and human serum:
FIGS. 27—1:0.5:0.5:1 ratio for Flr-anti-drug Fab:mAb2:mAb1:ligand; and
FIGS. 28—10:5:1:1 ratio for Flr-anti-drug Fab:mAb2:mAb1:ligand.

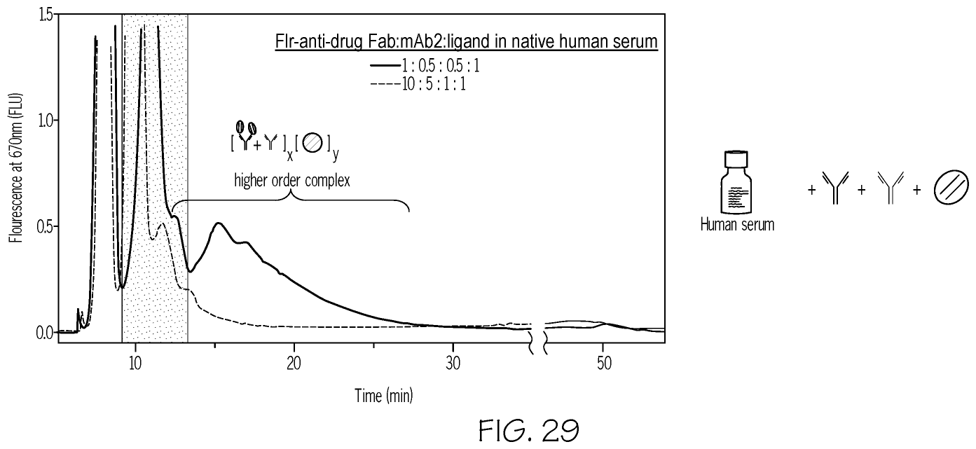

Flr-anti-drug Fab was demonstrated to be useful as an detection reagent to identify mAb2 containing higher order complex types in native human serum. Plot 1 used 1:0.5:0.5:1 Flr-anti-drug Fab:mAb2:mAb1:ligand Plot 2 used 10:5:1:1 Flr-anti-drug Fab:mAb2:mAb1:ligand. See FIG. 29.

The results demonstrated that A4F-Flr, in conjunction with A4F-MALS, can be used to characterize heterogeneous multi-antibody and ligand complexes in serum using three types of immune reagents, namely Flr-mAb2, Flr-anti-hFc Fab and Flr-anti-mAb2 Fab. Antibodies, such as mAb2, characterized according to the present invention were shown to form the same type of complexes in DPBS buffer, mouse serum, ligand-depleted human serum, and human serum.

### Example 4—Methods for Stability Assessment

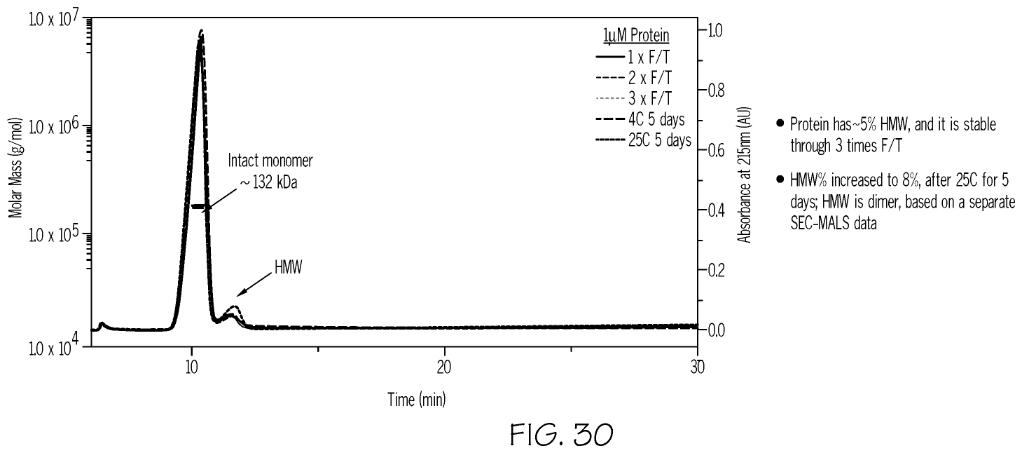

The inventions provide methods for stability assessment. FIG. 30 depicts data from an A4F-MALS analysis that a protein (scFv-GAA, which is single chain Fragment variable region bound to alpha glucosidase) is generally stable in a storage buffer (10 mM sodium phosphate (NaPi) buffer and pH 6.0) with freeze/thaw (F/T). More HMW species (3%) were observed after storage at 25° C. for 5 days.

Stability is typically tested at various storage temperature, such as refrigerated temperatures (about 1° C. to about 8° C.) and room temperatures (about 19° C. to about 25° C.). Stability at one or more temperatures can be assessed over periods of time, typically about 1 day to 1 year, and any amount of time within this range.

The scFv-GAA intact monomer had a measured molecular weight of 132 kilodaltons (kDa), and the expected molecular weight was 137 kDa, including glycosylation. In FIG. 30, the protein was tested with 1× freeze/thaw (F/T), 2× F/T and 3× F/T and shown to be stable, 3% more HMW was observed after storage at 25° C. for 5 days.

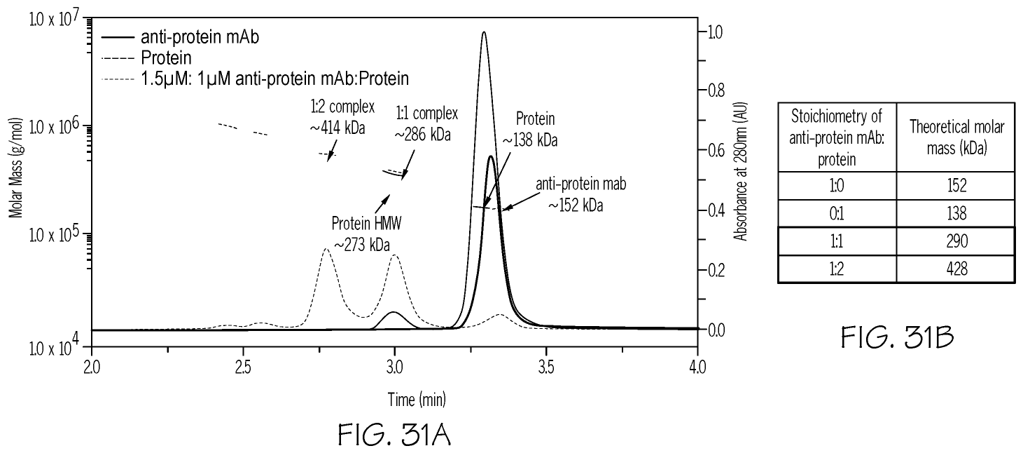

SEC-MALS showed that an anti-scFv-GAA monoclonal antibody can form canonical 1:1 and 1:2 complex types with the protein (svFv-GAA). See FIG. 31A. A chart showing stoichiometry of complex types and theoretical molar mass is provided at FIG. 31B. This experiment also could have been performed with A4F-MALS instead of SEC-MALS.

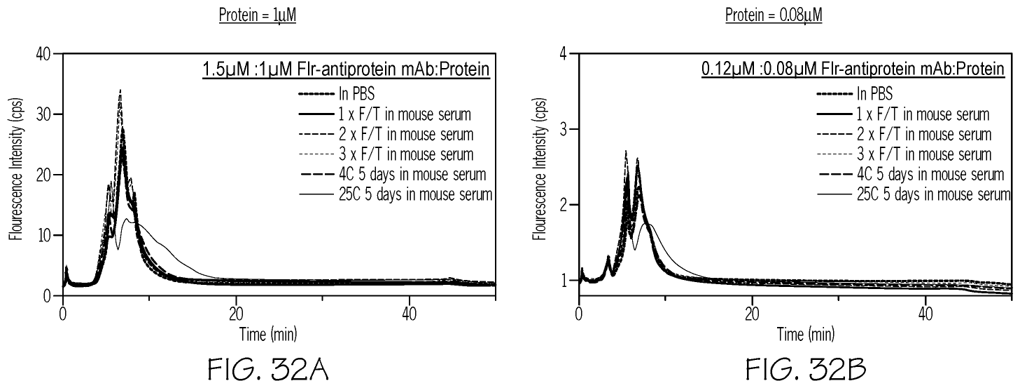

The A4F-Flr results show protein and Flr-anti-protein form the same type of complex in PBS buffer and mouse serum. FIG. 32A is a graph showing complex formation with 1 μM protein. FIG. 32B is a graph showing complex formation with 0.08 μM protein. At both concentrations, more high molecular weight (HWM) species were observed after storage at 25° C. for 5 days. 0.08 μM protein (FIG. 32B) showed less high molecular weight species than 1 μM protein (FIG. 32A).

The data show that (1) A4F-MALS combined with A4F-Flr, and optionally (2) SEC-MALS combined with SEC-Flr, according to the inventions are powerful approaches for evaluation and characterizing protein stability.

It is to be understood that the description, specific examples and data are given by way of illustration and are not intended to limit the present invention. Various changes and modifications within the present invention, including combining embodiments in whole and in part, will become apparent to the skilled artisan from the discussion, disclosure and data contained herein, and thus are considered part of the invention.

1. A method for characterizing one or more complex types comprising at least one protein of interest using asymmetric flow field-flow fractionation (A4F), wherein the method comprises the steps of:
(A) fractionating a first aliquot comprising complexes in a fractionation buffer using A4F, and determining at least one of molar mass, stoichiometry, and size distribution of the complexes in the first aliquot using Multi-Angle Light Scattering (MALS); and
(B) fractionating a second aliquot comprising the complexes bound with a fluorescence labeled detection reagent in a biological fluid using A4F, and detecting the protein of interest in the complexes using a fluorescence detector,
wherein (A) and (B) can be performed consecutively in any order or simultaneously; and
(C) comparing the elution time profiles from (A) and (B) to characterize complex types.

2. The method of claim 1, wherein the protein of interest is a monoclonal antibody, or an Fc-fusion protein.

3. (canceled)

4. The method according to claim 1, wherein the protein complexes are monoclonal antibody (mAb)—ligand complexes.

5. The method according to claim 1, wherein the second aliquot comprises protein complexes in serum.

6. The method according to claim 1, wherein the detection reagent is a fluorescence labeled antibody, a fluorescence labeled anti-Fc Fab or a fluorescence labeled anti-antibody Fab.

7. The method according to claim 1, wherein the method can:
determine stoichiometry of complexes in serum;
determine mass of complexes in serum; and
determine size distribution of complexes in serum.

8. (canceled)

9. (canceled)

10. The method according to claim 1, wherein the second aliquot is diluted in a fractionation buffer.

11. A method for evaluation stability of a protein in a biological fluid using asymmetric flow field-flow fractionation (A4F), wherein the method comprises the steps of:
(A) fractionating a first aliquot comprising the protein in a fractionation buffer using A4F, and determining size distribution of the protein in the first aliquot using Multi-Angle Light Scattering (MALS);
(B) fractionating a second aliquot comprising the protein bound with a fluorescence labeled detection reagent in a biological fluid using A4F, and detecting the protein using a fluorescence detector,
wherein (A) and (B) can be performed consecutively in any order or simultaneously; and
(C) comparing the elution time profiles from (A) and (B).

12. The method according to claim 11, wherein the detection reagent is a fluorescence labeled antibody, a fluorescence labeled anti-Fc Fab or a fluorescence labeled anti-antibody Fab.

13. The method according to claim 11, wherein the formation of high molecular weight species of the protein indicates instability.

14. The method according to claim 11, wherein the protein is subjected to one or more freeze-thaw cycles prior to conducting step (A).

15. The method according to claim 11, wherein the protein is stored prior to conducting step (A).

16. The method according to claim 15, wherein the protein is stored at 25° C.

17. The method according to claim 15, wherein the protein is stored for at least 5 days.

18. The method according to claim 11, wherein the second aliquot is diluted in a fractionation buffer.

19. A method for characterizing one or more complex types comprising at least one protein of interest using fractionation, wherein the method comprises the steps of:
(A) fractionating a first aliquot comprising the complexes in a fractionation buffer using asymmetric flow field-flow fractionation (A4F) or size exclusion chromatography (SEC), and determining at least one of molar mass, stoichiometry, and size distribution of the complexes in the first aliquot using Multi-Angle Light Scattering (MALS);
(B) fractionating a second aliquot comprising the complexes bound with a fluorescence labeled detection reagent in a biological fluid using A4F or SEC, and detecting the protein of interest in the complexes using a fluorescence detector,
wherein (A) and (B) can be performed consecutively in any order or simultaneously; and
(C) comparing the elution time profiles from (A) and (B) to characterize complex types.

20. The method according to claim 19, wherein steps (A) and (B) use A4F.

21. The method according to claim 20, wherein steps (A) and (B) use SEC.

22. The method according to claim 19, wherein the detection reagent is a fluorescent-labeled antibody, a fluorescent-labeled anti-Fc Fab or a fluorescent-labeled anti-antibody Fab.

23. The method according to claim 19, wherein the stability of the complexes can be determined.

24. The method according to claim 23, wherein the formation of high molecular weight species of the protein indicates instability.

25. The method according to claim 19, wherein the second aliquot is diluted in a fractionation buffer.

26. The method according to claim 19, wherein the protein of interest is a monoclonal antibody, or an Fc-fusion protein.

27. (canceled)

28. A system for performing the method described in claim 1.

29. A system for performing the method described in claim 11.

30. A system for performing the method described in claim 19.
